(19) United States
(12) Patent Application Publication
(10) Pub. No.: US 2017/0231926 A1
(43) Pub. Date: Aug. 17, 2017

(54) LIVESTOCK PRODUCTION METHOD AND LIVESTOCK-GROWTH PROMOTING METHOD

(71) Applicants: NISSHIN PHARMA INC., Chiyoda-ku (JP); KENKO CORPORATION, Chiyoda-ku (JP)

(72) Inventors: Toshihiko KURIKI, Chiyoda-ku (JP); Toshihiko OGURA, Chiyoda-ku (JP); Shinichi SONODA, Chiyoda-ku (JP); Taro MURAKAMI, Chiyoda-ku (JP)

(73) Assignees: NISSHIN PHARMA INC., Chiyoda-ku (JP); KENKO CORPORATION, Chiyoda-ku (JP)

(21) Appl. No.: 15/503,190

(22) PCT Filed: Aug. 13, 2014
(86) PCT No.: PCT/JP2014/071363

§ 371 Date: Feb. 10, 2017

(51) Int. Cl. A61K 31/122; A23K 50/75; A23K 20/189; A61K 47/10; A61K 47/36

(52) U.S. Cl. CPC A61K 31/122; A61K 47/10; A61K 47/36; A23K 20/189; A23K 50/75

(57) ABSTRACT

The present invention provides a method for producing livestock, comprising supplying coenzyme Q10-containing water to livestock, wherein the coenzyme Q10-containing water comprises coenzyme Q10 emulsified with a hydrophilic base material, and the hydrophilic base material does not comprise a synthetic surfactant.

### TECHNICAL FIELD

The present invention relates to a method for producing livestock at a high production efficiency.

### BACKGROUND ART

Livestock such as cattle, swine, sheep, and chicken are generally reared in a short period of time in a state of being densely accommodated in the barn. In particular, edible poultry typified by broilers are a breed that is improved so as to be grown in a short period of time, and for example, it takes from four to five months for full growth of chicken in nature but broilers can reach full growth in from 40 to 50 days. Meanwhile, livestock that are reared in a state of being densely accommodated in the barn undergo sudden death during rearing because of lack of exercise, stress, and disease, for example, in some cases. Furthermore, broilers are grown up in a short period of time so as to have a body weight of about from 2.5 to 3 kg, the heart thereof is thus likely to be burdened so that they are fallen into sudden death syndrome and often develop ascites, and over ten percent to several tens percent of them die before they reach full growth in some cases. Drugs such as antibiotics are used in order to prevent livestock from with diseases, but the use of drugs is accompanied by a problem such as reduction of the intestinal flora of livestock or a risk of emergence of resistant bacteria. In addition, there is an increasing demand for meat that is less exposed to drugs in recent years.

Coenzyme Q10 (hereinafter, also referred to as CoQ10 in some case in the present specification) is a coenzyme for an electron transport system and is also known as a vitamin-like substance having an action to improve the oxygen utilization efficiency. Hitherto, feed for livestock containing coenzyme Q10 has been known. In Patent Literature 1, it is described that it is possible to favorably grow livestock or improve intestinal flora of livestock or increase the feed efficiency by administering feed containing therein coenzyme Q10 to livestock with reduced or no antibiotics. In Patent Literatures 2 and 3, it is described that coenzyme Q9 or coenzyme Q10 is effective in the prevention of ascites or sudden death syndrome in broilers. In Patent Literature 4, it is described that feed composition for poultry containing coenzyme Q and a substance selected from an antioxidant, an antacid, an ammonia generation inhibitor has an effect of preventing ascites or sudden death syndrome in broilers and improving the rearing rate thereof.

Meanwhile, coenzyme Q10 is poorly soluble in water, and thus it floats on water surface or precipitates so as not to be readily dispersed in water when it is simply added to water or even when it is mixed with water after mixing with a powder or fat. Hence, it has been hitherto difficult to administer coenzyme Q10 in an accurate amount. In addition, the coenzyme Q10-containing feed described above is required to be administered for a long period of time in order to obtain a desired disease-preventing effect or rearing rate-improving effect.

In order to cope with this problem, in Patent Literature 5, water-dispersible ascites inhibitor for poultry containing coenzyme Q and a surfactant is provided. In Patent Literature 5, it is also described that it is possible to decrease the occurrence of ascites by administration of the inhibitor for a shorter period of time than conventional, as a result of administration (for 8 days) of the inhibitor to broilers from 33 days of age to 40 days of age as it is added to the drinking water.

In Patent Literature 6, it is described that coenzyme Q10 has an effect such as stabilization or antioxidation of biological membranes and is effective in symptom improvement of angina pectoris, heart failure, ischemic heart disease, muscular dystrophy, or prevent ion and treatment of hypertension, arteriosclerosis, heart disease, diabetes, and periodontal disease, prevention of side effect by anticancer or psychotropic drugs, and fatigue recovery or motor function recovery, for example. In addition, in Patent Literature 6, it is described that a coenzyme Q10-containing liquid composition is obtained by dispersing and emulsifying coenzyme Q10 with an aqueous liquid containing a water soluble substance composed of octenyl succinate starch and dextrin and glycerin and exhibits excellent stability and bioavailability.

### CITATION LIST

### Patent Literature

Patent Literature 1: JP 2011-182748 A
Patent Literature 2: JP 06-287136 A
Patent Literature 3: JP 7-123928 A
Patent Literature 4: JP 9-187229 A
Patent Literature 5: WO 2003/007928 A
Patent Literature 6: WO 2006/022187 A

### SUMMARY OF INVENTION

### Technical Problem

A decrease in death rate due to sudden death and diseases during the rearing period of livestock, an increase in body weight gain rate of livestock, and a decrease in feed consumption rate (FCR) are required.

### Solution to Problem

The present inventors have found that it is possible to significantly decrease the death rate due to sudden death and diseases during the rearing period of livestock and further to promote the growth of livestock to thereby decrease the feed consumption rate (FCR) by feeding water which does not comprise a synthetic surfactant but comprises coenzyme Q10 emulsified with a hydrophilic base material to livestock as drinking water.

That is, the present invention provides a method for producing livestock, comprising supplying coenzyme Q10-containing water to livestock, wherein the coenzyme Q10 -containing water comprises coenzyme Q10 emulsified with a hydrophilic base material, and the hydrophilic base material does not comprise a synthetic surfactant.

In addition, the present invention provides a method for promoting the growth of livestock, comprising supplying coenzyme Q10-containing water to livestock, wherein the coenzyme Q10-containing water comprises coenzyme Q10 emulsified with a hydrophilic base material, and the hydrophilic base material does not comprise a synthetic surfactant.

In addition, the present invention provides a method for preventing ascites or sudden death syndrome in livestock, comprising supplying coenzyme Q10-containing water to livestock, wherein the coenzyme Q10-containing water comprises coenzyme Q10 emulsified with a hydrophilic base material, and the hydrophilic base material does not comprise a synthetic surfactant.

Furthermore, the present invention provides use of coenzyme Q10-containing water for the production of drinking water for livestock (here, the coenzyme Q10-containing water comprises the coenzyme Q10 emulsified with a hydrophilic base material and the hydrophilic base material does not comprise a synthetic surfactant).

Furthermore, the present invention provides use of coenzyme Q10-containing water for the production of a growth promoter for livestock (here, the coenzyme Q10-containing water comprises the coenzyme Q10 emulsified with a hydrophilic base material and the hydrophilic base material does not comprise a synthetic surfactant).

Furthermore, the present invention provide use of coenzyme Q10-containing water for the production of an ascites or sudden death syndrome inhibitor for livestock (here, the coenzyme Q10-containing water comprises the coenzyme Q10 emulsified with a hydrophilic base material and the hydrophilic base material does not comprise a synthetic surfactant).

Furthermore, the present invention provides coenzyme Q10-containing water for use in drinking water for livestock (here, the coenzyme Q10-containing water comprises the coenzyme Q10 emulsified with a hydrophilic base material and the hydrophilic base material does not comprise a synthetic surfactant).

Furthermore, the present invention provides coenzyme Q10 -containing water for use in the promotion of livestock growth (here, the coenzyme Q10-containing water comprises the coenzyme Q10 emulsified with a hydrophilic base material and the hydrophilic base material does not comprise a synthetic surfactant).

Furthermore, the present invention provides coenzyme Q10-containing water for use in the prevention of ascites or sudden death syndrome in livestock (here, the coenzyme Q10-containing water comprises the coenzyme Q10 emulsified with a hydrophilic base material and the hydrophilic base material does not comprise a synthetic surfactant).

In an embodiment of the present invention, the livestock are poultry.

In an embodiment of the present invention, the poultry are a broiler.

In an embodiment of the present invention, the coenzyme Q10-containing water is supplied to livestock from one day of age to 32 days of age.

In an embodiment of the present invention, the coenzyme Q10-containing water is supplied to livestock in an amount of from 0.01 to 20 mg/kg of body weight per day in terms of an amount of CoQ10.

In an embodiment of the present invention, the hydrophilic base material is two or more selected from the group consisting of hydrophilic modified starch, gum exhibiting an emulsifying property, water soluble cellulose, dextrin, cyclodextrin, polyhydric alcohol, fructose, glucose, and casein.

In an embodiment of the present invention, the hydrophilic base material comprises octenyl succinate starch, dextrin, and glycerin.

In an embodiment of the present invention, the coenzyme Q10 emulsified with a hydrophilic base material comprises coenzyme Q10 at from 3 to 80 mass %, glycerin at from 0.01 to 25 mass %, and octenyl succinate starch and dextrin at from 19 to 96 mass % in total in terms of dry mass.

In an embodiment of the present invention, a mass ratio of the coenzyme Q10 to the hydrophilic base material in the coenzyme Q10 emulsified with a hydrophilic base material is from 20:80 to 60:40 in terms of dry mass.

In an embodiment of the present invention, the coenzyme Q10 emulsified with a hydrophilic base material is particles having an average particle size of 15 μm or less.

### Advantageous Effects of Invention

According to the present invention, it is possible to significantly decrease the death rate of livestock during the rearing period until the full growth and further to decrease the feed consumption rate (FCR) due to promotion of the growth of livestock. According to the present invention, it is possible to improve the production efficiency of livestock.

### MODE FOR CARRYING OUT THE INVENTION

In the present specification, livestock refers to an arbitrary animal that is reared for the production of edible or useful substance. Examples of the livestock may include livestock mammals such as cattle, swine, and sheep and poultry such as chicken, turkey, duck, goose, quail, pheasant, and guinea fowl.

The coenzyme Q10 -containing water to be used in the present invention is water containing coenzyme Q10 that is emulsified with a hydrophilic base material (hereinafter, referred to as water-soluble type CoQ10 in some cases).

Examples of the hydrophilic base material contained in the water-soluble type CoQ10 may include water soluble polysaccharides including, for example, a hydrophilic modified starch such as octenyl succinate starch and hydroxypropyl starch, gum exhibiting an emulsifying property such as gum Arabic and xanthan gum, water soluble cellulose such as methylcellulose, ethylcellulose and hydroxypropylcellulose, agar, water soluble corn fiber, carmellose sodium, polyvinyl pyrrolidone, dextrin, cyclodextrin and the like; polyhydric alcohol including glycerin and the like; fructose; glucose; casein; and gelatin, and two or more, preferably three or more selected from these are used. Examples of a preferred combination of hydrophilic base materials may include a combination comprising hydrophilic modified starch such as octenyl succinate starch or hydroxypropyl starch, dextrin, and polyhydric alcohol such as glycerin, more preferably a combination comprising at least octenyl succinate starch, dextrin, and glycerin, even more preferably a combination consisting of octenyl succinate starch, dextrin, and glycerin.

The hydrophilic base material does not comprise a surfactant. In addition, the water-soluble type CoQ10 does not comprise a surfactant. Here, the surfactant generally refers to a synthetic surfactant produced by a chemical synthesis, and examples thereof may include an anionic synthetic surfactant such as a monoalkyl sulfate, an alkyl polyoxyethylene sulfate, an alkylbenzene sulfonate, or a monoalkyl phosphate; a cationic synthetic surfactant such as an alkyl trimethyl ammonium salt, a dialkyl dimethyl ammonium salt, or an alkyl benzyl dimethyl ammonium salt, an amphoteric synthetic surfactant such as an alkyl dimethyl amine oxide or an alkyl carboxyl betaine; and a nonionic synthetic surfactant such as a polyoxyethylene alkyl ether, a fatty acid sorbitan ester, a polyoxyethylene fatty acid ester, a polyoxyethylene sorbitan fatty acid ester, a polyhydric alcohol fatty acid ester, a glycerin fatty acid ester, a polyglycerin fatty acid ester alkyl polyglucoside, a fatty acid diethanol amide, or an alkyl monoglyceryl ether.

As means for preparing water-soluble type CoQ10, it is possible to employ a known method for preparing CoQ10 into a solid or liquid emulsion together with the hydrophilic base material. For example, it is possible to employ a method to prepare an emulsion having a desired particle size by suspending CoQ10 and a hydrophilic base material in water and then stirring or subjecting the suspension to high-pressure emulsification. Preferred examples thereof may include a method in which an aqueous solution of the hydrophilic base material is prepared in the presence of an organic acid, and CoQ10 subjected to thermal melting is introduced into this and dispersed and emulsified by using, for example, a high-pressure homogenizer to obtain an emulsion (e.g., refer to JP 2003-055203 A) or a method in which an aqueous solution of the hydrophilic base material containing glycerin is prepared, the solution is heated in advance, and CoQ10 subjected to thermal melting is introduced into this and dispersed and emulsified by using, for example, a high-pressure homogenizer to obtain an emulsion (e.g., refer to Patent Literature 6). If necessary, a liquid containing the emulsion may be dried and powdered by a usual method.

Preferred examples of the water-soluble type CoQ10 to be used in the present invent ion may include an emulsion composition which comprises CoQ10 at from 3 to 80 mass %, glycerin at from 0.01 to 25 mass %, and octenyl succinate starch and dextrin at from 19 to 96 mass % in total in terms of dry mass.

Upon the production of the water-soluble type CoQ10, it is possible to further add an organic acid to an aqueous solution before or after dispersing and emulsifying CoQ10 in the aqueous solution for the purpose of stabilizing CoQ10. Examples of the organic acid may include citric acid, succinic acid, fumaric acid, lactic acid, gluconic acid, malic acid, tartaric acid, a salt thereof, and a mixture thereof. Examples of the salt of the organic acids may include a sodium salt, a potassium salt, a magnesium salt, and a calcium salt of the organic acids. The amount of the organic acid added varies depending on the kind of the organic acid, but it is generally in a range of from 0.01 to 30 mass %, preferably in a range of from 0.05 to 15 mass %, in the entire amount of the water-soluble type CoQ10 as a dry mass. Hence, another preferred example of the water-soluble type CoQ10 to be used in the present invention may include an emulsion composition which comprises CoQ10 at from 3 to 80 mass %, glycerin at from 0.01 to 25 mass %, octenyl succinate starch and dextrin at from 19 to 96 mass % in total, and an organic acid at from 0.01 to 30 mass % in terms of dry mass.

More preferably, the mass ratio of CoQ10 to the hydrophilic base material in the water-soluble type CoQ10 is from 20:80 to 60:40 in terms of dry mass. The form of the water-soluble type CoQ10 maybe a liquid emulsion or a solid or powdery emulsion. As the water-soluble type CoQ10 which does not comprise a surfactant and is used in the present invention, a commercially available product (e.g., Aqua Q10-P40 manufactured by Nisshin Pharma Inc.) can be used.

The water-soluble type CoQ10 is easily dispersed in water since it is an emulsion. Hence, it is possible to produce the coenzyme Q10-containing water to be used in the present invention by adding the water-soluble type CoQ10 to water and stirring the mixture. Alternatively, the water-soluble type CoQ10 may be used as the coenzyme Q10-containing water as it is in a case in which the water-soluble type CoQ10 is a liquid emulsion. The kind of water to be used in the production of the coenzyme Q10-containing water is not particularly limited, and it may be purified water or tap water. Preferably, the coenzyme Q10-containing water to be used in the present invention is a substantially homogeneous aqueous emulsion solution.

The emulsified CoQ10 contained in the coenzyme Q10-containing water is preferably particles having an average particle size of 15 μm or less. There is a case in which the CoQ10 particles may aggregate or precipitate in the coenzyme Q10-containing water or adhere to, for example, the water supply container when the average particle size of the CoQ10 particles exceeds 15 μm. The average particle system of the CoQ10 particles is more preferably 5 μm or less, even more preferably 1 μm or less. In the present specification, the average particle size of particles refers to the volume mean diameter (MV) of particle calculated by a laser diffraction and scattering method.

In the present invention, the coenzyme Q10-containing water is supplied to livestock as drinking water. The emulsified CoQ10 contained in the coenzyme Q10-containing water is administered to the livestock as the livestock drink water. The procedure to supply the coenzyme Q10-containing water to the livestock may conform to a usual procedure to supply water to the livestock.

In the coenzyme Q10 -containing water, the emulsified CoQ10 particles are substantially homogeneously dispersed without precipitating or floating on water surface, and thus the dosage of CoQ10 to the livestock to which the coenzyme Q10-containing water is supplied is proportional to the intake of the coenzyme Q10-containing water by the livestock. Hence, according to the present invention, it is possible to control the dosage of CoQ10 to the livestock by controlling the CoQ10 concentration in the coenzyme Q10-containing water and the water intake by the livestock.

CoQ10 contained in the coenzyme Q10-containing water is highly soluble in water since it is emulsified, and thus it exhibits high bioavailability (e.g., absorbability and biological utilization rate) even when being orally taken. Hence, the livestock to which the coenzyme Q10-containing water is supplied can sufficiently have a physiological action of CoQ10 Particularly in the livestock to which the coenzyme Q10-containing water is supplied, the body weight gain rate increases as the growth is promoted but the feed consumption rate (FCR) decreases.

In the present invention, the livestock to which the coenzyme Q10-containing water is supplied may be any of the livestock exemplified above, but the livestock are preferably poultry, more preferably chickens, even more preferably broilers The coenzyme Q10-containing water is effective in prevention of ascites and sudden death syndrome in poultry such as broilers and can significantly decrease the death rate during the rearing period of broilers.

The amount of the coenzyme Q10-containing water supplied to the livestock is preferably from 0.01 to 20 mg/kg of body weight, more preferably from 0.05 to 10 mg/kg of body weight, even more preferably from 1 to 10 mg/kg of body weight per day in terms of the amount of CoQ10. In other words, the amount of the coenzyme Q10-containing water supplied to the livestock is adjusted so that the dosage of CoQ10 to the livestock is preferably from 0.01 to 20 mg/kg of body weight, more preferably from 0.05 to 10 mg/kg of body weight, even more preferably from 1 to 10 mg/kg of body weight per day.

The CoQ10 concentration in the coenzyme Q10-containing water to be supplied to the livestock may be preferably from 0.001 to 0.1 mass/vol %, more preferably 0. 005 to 0.05 mass/vol %, even more preferably from 0.008 to 0.0.03 mass/vol %. Preferably, CoQ10 concentration in the coenzyme Q10-containing water is determined depending on the average water intake by the livestock and the dosage of CoQ10 to be required.

In the present invention, the coenzyme Q10-containing water is preferably supplied to the immature (before reproductive stage) livestock. In the case of broilers, the coenzyme Q10-containing water is supplied to the broilers preferably for a period of from one day of age to 32 days of age, more preferably for a period of from five days of age to 25 days of age, even more preferably for a period of from eight days of age to 22 days of age.

### EXAMPLES

Hereinafter, the present invention will be specifically described with reference to Examples, but the present invention is not limited to the following Examples.

### Reference Example 1

### Preparation of Coenzyme Q10-Containing Water

Starch sodium octenyl succinate, dextrin, and glycerin were added to purified water, and the resultant mixture was heated to about 60° C. CoQ10 was mixed with this and further emulsified by allowing the resultant mixture to pass through a high-pressure homogenizer, thereby obtaining a homogenous emulsion. Subsequently, the emulsion was jetted into hot air to remove the moisture, thereby obtaining a powder composition. The powder composition thus obtained had the following composition.

| | |
|---|---|
| Coenzyme Q10 | 42% |
| Octenyl succinate starch | 40% |
| Dextrin | 15% |
| Glycerin | 3% |

A homogeneous emulsion was produced by adding the powder composition to water and stirring. The average particle size of CoQ10 in the emulsion was measured by using a laser diffraction particle size measuring instrument, and the result was 0.2 μm. The powder composition was added to water so as to be 0.01% (w/v) of an amount of CoQ10, thereby preparing the coenzyme Q10-containing water, and this was used in the following Example.

### Example 1

Broilers were reared from hatching (0 day of age) to 42 days of age. The rearing conditions were that the ventilation was conducted by using a fan, the temperature (26° C.) was kept by using an air heater, the light was turned on for 24 hours, and the rearing density was adjusted to 0.85 birds/ft² or more. As the feed, the broilers were allowed to freely take normal feed tailored to the growth stage thereof. The broilers were allowed to freely take drinking water, but the coenzyme Q10-containing water prepared in Reference Example 1 (0.01% (w/v) CoQ10) was supplied to the broilers during a period of from eight days of age to 22 days of age.

The intake of feed and drinking water was daily measured and the dosage of CoQ10 was determined from the water intake.

The body weight of each of the individuals at the stage of from zero day of age to 42 days of age was measured and the amount (kg) of increase in body weight during the rearing period was determined.

The feed consumption (kg) was divided by the amount (kg) of increase in body weight to calculate the feed consumption rate (FCR).

The number of surviving individuals was daily measured and the survival rate (%) was determined at the stage of 42 days of age.

The test described above was conducted two times. The number of samples in each test was 21200 birds for the first time and 21800 birds for the second time. The results of the test are presented in Table 1.

TABLE 1

| | First time | Second time |
|---|---|---|
| Number of samples | 21200 | 21800 |
| Dosage of CoQ10 | 5.66 mg/ | 5.50 mg/ |
| | day/individual | day/individual |
| Survival rate (%) | 98.43 | 98.95 |
| Amount of increase in | 2.5 | 2.55 |
| body weight (kg) | | |
| FCR | 1.9 | 1.87 |

1. A method for producing livestock, the method comprising:
supplying coenzyme Q10-containing water to livestock, wherein
the coenzyme Q10-containing water comprises coenzyme Q10 emulsified with a hydrophilic base material, and the hydrophilic base material does not comprise a synthetic surfactant.

2. The method according to claim 1, wherein the livestock are poultry.

3. The method according to claim 2, wherein the poultry are a broiler.

4. The method according to claim 1, wherein the coenzyme Q10-containing water is supplied to the livestock from one day of age to 32 days of age.

5. The method according to claim 1, wherein the coenzyme Q10-containing water is supplied to the livestock in an amount of from 0.01 to 20 mg/kg of body weight per day in terms of an amount of coenzyme Q10.

6. The method according to claim 1, wherein the hydrophilic base material is two or more selected from the group consisting of hydrophilic modified starch, gum exhibiting an emulsifying property, water soluble cellulose, dextrin, cyclodextrin, polyhydric alcohol, fructose, glucose, and casein.

7. The method according to claim 1, wherein the hydrophilic base material comprises octenyl succinate starch, dextrin, and glycerin.

8. The method according to claim 1, wherein the coenzyme Q10 emulsified with a hydrophilic base material comprises coenzyme Q10 at from 3 to 80 mass %, glycerin at from 0.01 to 25 mass %, and octenyl succinate starch and dextrin at from 19 to 96 mass % in total in terms of dry mass.

9. The method according to claim 1, wherein a mass ratio of the coenzyme Q10 to the hydrophilic base material in the coenzyme Q10 emulsified with a hydrophilic base material is from 20:80 to 60:40 in terms of dry mass.

10. The method according to claim 1, wherein the coenzyme Q10 emulsified with a hydrophilic base material is in the form of particles having an average particle size of 15 μm or less.

11. A method for promoting the growth of livestock, the method comprising:
supplying coenzyme Q10-containing water to livestock, wherein
the coenzyme Q10-containing water comprises coenzyme Q10 emulsified with a hydrophilic base material, and the hydrophilic base material does not comprise a synthetic surfactant.

12. The method according to claim 11, wherein the livestock are poultry.

13. The method according to claim 12, wherein the poultry are a broiler.

14. The method according to claim 11, wherein the coenzyme Q10-containing water is supplied to the livestock from one day of age to 32 days of age.

15. The method according to claim 11, wherein the coenzyme Q10-containing water is supplied to the livestock in an amount of from 0.01 to 20 mg/kg of body weight per day in terms of an amount of coenzyme Q10.

16. The method according to claim 11, wherein the hydrophilic base material is two or more selected from the group consisting of hydrophilic modified starch, gum exhibiting an emulsifying property, water soluble cellulose, dextrin, cyclodextrin, polyhydric alcohol, fructose, glucose, and casein.

17. The method according to claim 11, wherein the hydrophilic base material comprises octenyl succinate starch, dextrin, and glycerin.

18. The method according to claim 11, wherein the coenzyme Q10 emulsified with a hydrophilic base material comprises coenzyme Q10 at from 3 to 80 mass %, glycerin at from 0.01 to 25 mass %, and octenyl succinate starch and dextrin at from 19 to 96 mass % in total in terms of dry mass.

19. The method according to claim 11, wherein a mass ratio of the coenzyme Q10 to the hydrophilic base material in the coenzyme Q10 emulsified with a hydrophilic base material is from 20:80 to 60:40 in terms of dry mass.

20. The method according to claim 11, wherein the coenzyme Q10 emulsified with the hydrophilic base material is in the form of particles having an average particle size of 15 μm or less.

21. A method for preventing ascites or sudden death syndrome in livestock, the method comprising:
supplying coenzyme Q10-containing water to livestock, wherein
the coenzyme Q10-containing water comprises coenzyme Q10 emulsified with a hydrophilic base material, and the hydrophilic base material does not comprise a synthetic surfactant.

22. The method according to claim 21, wherein the livestock are poultry.

23. The method according to claim 22, wherein the poultry are a broiler.

24. The method according to claim 21, wherein the coenzyme Q10-containing water is supplied to the livestock from one day of age to 32 days of age.

25. The method according to claim 21, wherein the coenzyme Q10-containing water is supplied to the livestock in an amount of from 0.01 to 20 mg/kg of body weight per day in terms of an amount of coenzyme Q10.

26. The method according to claim 21, wherein the hydrophilic base material is two or more selected from the group consisting of hydrophilic modified starch, gum exhibiting an emulsifying property, water soluble cellulose, dextrin, cyclodextrin, polyhydric alcohol, fructose, glucose, and casein.

27. The method according to claim 21, wherein the hydrophilic base material comprises octenyl succinate starch, dextrin, and glycerin.

28. The method according to claim 21, wherein the coenzyme Q10 emulsified with a hydrophilic base material comprises coenzyme Q10 at from 3 to 80 mass %, glycerin at from 0.01 to 25 mass %, and octenyl succinate starch and dextrin at from 19 to 96 mass % in total in terms of dry mass.

29. The method according to claim 21, wherein a mass ratio of the coenzyme Q10 to the hydrophilic base material in the coenzyme Q10 emulsified with a hydrophilic base material is from 20:80 to 60:40 in terms of dry mass.

30. The method according to claim 21, wherein the coenzyme Q10 emulsified with a hydrophilic base material is in the form of particles having an average particle size of 15 μm or less.
